(19) United States
(12) Patent Application Publication
(10) Pub. No.: US 2026/0108648 A1
(43) Pub. Date: Apr. 23, 2026

(54) SCREEN BASKET, METHOD FOR GRIPPING A SCREEN BASKET AND SYSTEM

(71) Applicant: Aesculap AG, Tuttlingen (DE)

(72) Inventors: Arne Jansen-Troy, Stockach (DE); Leo Roempp, Hardt (DE); Johannes Hermann, Schoemberg (DE); Tobias Koller, Tuttlingen (DE)

(21) Appl. No.: 19/361,190

(22) Filed: Oct. 17, 2025

(30) Foreign Application Priority Data
Oct. 18, 2024 (DE) 10 2024 130 348.4

(51) Int. Cl. A61L 2/26; A61L 2/18; A61L 2/24

(52) U.S. Cl. CPC A61L 2/26; A61L 2/18; A61L 2/24; A61L 2103/15; A61L 2202/14

(57) ABSTRACT

A screen basket has a screen basket body and a screen basket positioning element for positioning and facilitating gripping of the screen basket. The screen basket also has a storage space for flushing sterile goods and a protective space separated from the storage space by a partition wall. A gripper can dip within the protective space and grip behind a supporting part of the screen basket.

### CROSS-REFERENCE TO RELATED APPLICATION

This application claims priority under 35 U.S.C. § 119 to German Application No. 10 2024 130 348.4, filed on Oct. 18, 2024, the content of which is incorporated by reference herein in its entirety.

### FIELD

The present disclosure relates to a screen basket with a screen basket positioning element. Furthermore, the present disclosure relates to a system comprising the screen basket and the screen basket positioning element and a method for gripping the screen basket.

### BACKGROUND

DE 10 2020 131 104 A1 discloses a sterilization tray with a fixing handle, which is intended and designed for insertion into and removal from a container tray.

DE 10 2020 103 131 A1 discloses a labeling system for sterile containers and screen trays.

DE 10 2018 104 942 A1 discloses a sterilization tray with a handle that can be moved into the removal position by means of an actuation.

### SUMMARY

The task of the present disclosure is to create a screen basket which can be gripped, in particular automatically, by means of a gripper.

The present disclosure relates to a screen basket with a screen basket positioning element for positioning and facilitating gripping of the screen basket, which has a storage space for flushing sterile goods and a protective space separated therefrom by means of a partition wall, within which a gripper can dip in and grip behind a support part of the screen basket.

The screen basket positioning element is used mechanically to position a screen basket in a sterile goods container more precisely or to severely restrict its movement. The screen basket positioning element can be positively and/or non-positively attached to a free edge section of the screen basket. The positioning element is also used for tracking and tracing with the aid of optical or machine-readable markings for navigation and information comparison with stored data.

Protective space prevents an instrument or part of it from lying at a point on the screen basket where the gripper is immersed. An instrument could reach such a point if the instruments are stored chaotically in the strainer basket or if the instruments shift during transportation. There are certain screen basket configurations in which some or all instruments are assigned a defined storage position or a specific storage location and in which the instruments stored in this way are held or secured relatively securely against unintentional displacement. However, the majority of screen baskets do not have a specific storage position for all recorded instruments or even for any recorded instrument. In these cases, the instruments are basically deposited in the screen basket like bulk material in bulk, without occupying a defined position or orientation and are therefore not secured against unintentional displacement. In any case, the protective space prevents the gripper from colliding with instruments when it is immersed or retracted into the screen basket. Thus, the protective space prevents the instruments, the screen basket or an automated, in particular robotic gripping system of the gripper from being damaged. In addition, protective space prevents dirt or consumables from entering the working area of the gripper.

The protective space is preferably open at the top but offers protection to the lower side and at least three other sides that are not already covered by the edge of the screen basket. The protective space is preferably only large enough for the gripper to enter from above and move horizontally in the screen basket under the support and lift out the screen basket.

In one embodiment, it is provided that the screen basket positioning element is primarily attached to an edge section of the screen basket, in particular plugged in. The protective space can thus be designed in such a way that the screen basket positioning element can be attached to the screen basket without tools. The screen basket positioning element can be made of plastic, in particular as an injection-molded part.

The screen basket can be gripped in particular by a robot whose gripper of the gripping system grips behind or underneath curved bearing lugs. These bearing lugs can be arranged on the inside of the screen basket and enclose the free ends of a handlebar bent from a round steel and support them in a rotating manner. In this respect, in one embodiment it may be provided that the support part of the screen basket comprises the two bearing lugs in which the ends of the common handlebar of the screen basket are pivotably accommodated.

In one embodiment, it is provided that the partition wall has lower wall parts which, with respect to
a horizontal flushing position of the screen basket, are each arranged below the lower edges of the bearing lugs and are
spaced from the outer contours of the bearing lugs in horizontal directions pointing away from the edge section and inwards in such a way that the screen basket positioning element can be placed on the edge section from above without the lower wall parts coming into contact with the bearing lugs.

In other words, the screen basket positioning element can be attached to the screen basket from above in a straight movement without having to bend open any part of the partition wall.

In one embodiment, the partition wall is provided with intermediate partition wall parts that are arranged between the bearing lugs.

In one embodiment, it is provided that intermediate partition wall parts are connected to each other by means of a connecting wall.

In one embodiment, it is provided that the partition wall has inner partition wall parts which, with respect to a horizontal flushing position of the screen basket, are spaced so far away from the storage lugs in a horizontal direction pointing perpendicularly away from the edge section and towards the storage space that horizontal arms of the gripper or the grippers can dip below the lower edges of the storage lugs.

In one embodiment, it is provided that the inner partition wall parts have recesses in which the handlebar is partially received and supported on the inner partition wall part when the handlebar is in a horizontal position.

In one embodiment, it is provided that the partition wall has at least one lateral connecting part which connects one of the inner partition wall parts to one of a plurality of surfaces on which a further screen basket can be deposited.

In one embodiment, it is provided that the lower wall part has a machine-readable marking, which is designed in particular as a pattern. Such machine-readable marking is particularly visually recognizable, for example by means of a camera. Such a machine-readable marking is preferably attached to the bottom of the protective space in a horizontal position so that this marking is visible in a top view. This marking enables the gripping system, for example, to use a robot guidance camera to redundantly check whether objects are in the working or travel path of the gripper. This marking can be clearly formed as a defined pattern. If the pattern is not fully captured because there is an object in the work area, the automated gripping system does not move to a gripping position and reports an error to a control device via an interface. These machine-readable markings can be printed or already incorporated into or applied to the surface of the lower wall section by an injection molding process.

In a further embodiment, a machine-readable interface for detecting a position of the screen basket is provided outside the protective chamber.

The present disclosure further relates to a method for gripping the screen basket within a container, wherein an arm of the gripper is moved from above into the protective space and under the support part and is then lifted to lift out the screen basket.

In one embodiment, the method is designed in such a way that the gripper is first moved vertically downwards and then horizontally under the support part.

In one embodiment, the method is implemented in such a way that an optical detection device coupled with the gripper detects an area within the protective space in which a machine-readable marking is located, wherein an error is reported to a control device if the machine-readable marking cannot be detected or cannot be detected sufficiently.

In one embodiment, the method is designed in such a way that the control device causes the gripper
not to move into the protective space or
to stop a movement towards the protective space or
to move out of the protective space again after the error has been reported.

The present disclosure further relates to a system comprising the screen basket and the screen basket positioning element, wherein the container comprises a tub for receiving the screen basket, wherein the screen basket with the screen basket positioning element is insertable into the tub of the container and is adapted such that the mechanical interface restricts the movement of the screen basket

Preferably, the device has a surface comprising the machine-readable interface for recognizing the screen basket and/or the machine-readable interface for recognizing the position of the screen basket.

In one embodiment, the machine-readable interface for recognizing the screen basket and/or the machine-readable interface for recognizing the position of the screen basket and/or orientation is embossed into the surface or printed on or otherwise attached to the surface. This device is particularly easy to mass-produce.

In one embodiment, the surface is designed for stacking another screen basket. This means that several screen baskets can be arranged on top of each other.

Preferably, the machine-readable interface for recognizing the screen basket comprises an optically readable marker, in particular a barcode, a data matrix code, alphanumeric data or a QR code, which encodes a type identification of the screen basket in a machine-readable manner.

In one embodiment, the machine-readable interface for recognizing the position and/or orientation comprises a symbol with a unique indication of an orientation of the symbol. This allows an unmistakable indication of orientation.

It may be provided that the screen basket positioning element is made at least partly of plastic or at least partly of metal.

It may be provided that the screen basket positioning element comprises a transmitter which is designed to transmit an electromagnetic signal, in particular a Bluetooth Low Energy signal or a Radio Frequency Identification signal, for recognizing the screen basket, wherein the machine-readable interface for recognizing the screen basket comprises the transmitter and/or that the device comprises a transmitter which is designed to transmit an electromagnetic signal, in particular a Bluetooth Low Energy signal or a Radio Frequency Identification signal, for recognizing the position and/or orientation of the screen basket, wherein the machine-readable interface for recognizing the position and/or orientation of the screen basket comprises the transmitter.

It may be provided that the transmitter is glued to the screen basket positioning element and fixed in a force-fit or form-fit manner, or that the screen basket positioning element is at least partially made of plastic, with the transmitter being surrounded by the plastic.

### BRIEF DESCRIPTION OF THE DRAWINGS

Further advantageous embodiments can be seen in the following description and the drawing drawings.

FIG. 1 shows a perspective illustration of a screen basket positioning element;

FIG. 2 shows a view of a screen basket with a screen basket positioning element and a gripper;

FIG. 3a shows a section of the screen basket in detail B of FIG. 2;

FIG. 3b shows sectional view along the stepped line A-A of FIG. 2;

FIG. 4 shows a perspective view of the screen basket with the screen basket positioning element and the gripper; and

FIG. 5 shows a perspective illustration of a screen basket with two screen basket positioning elements inserted into a sterile goods container.

### DETAILED DESCRIPTION

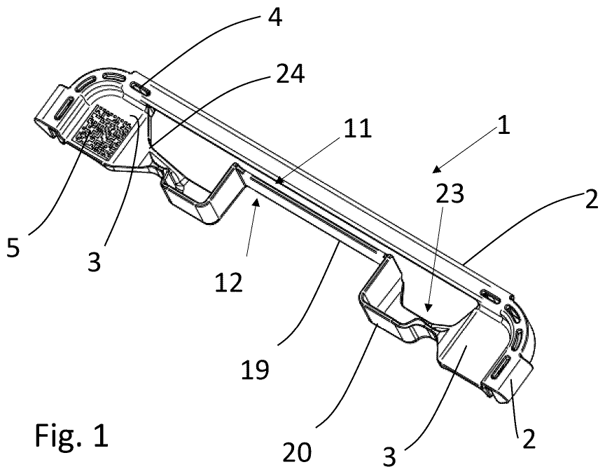

A screen basket positioning element 1 is shown in FIG. 1. The screen basket positioning element 1 is intended for positioning a screen basket in a container. Such a screen basket preferably comprises two screen basket positioning elements 1. In the example, the container is a sterile goods container.

Subsequent designations such as “top”, “bottom” refer to a horizontal flushing position of the screen basket, i.e. a position in which instruments inside the screen basket are flushed with a sterilization liquid, the surface of the sterilization liquid being parallel to an upper edge of the screen basket and the sterile goods container.

Subsequent designations such as “outside”, “inside” refer to the screen basket. This applies in particular to directions “inwards” and “outwards”, which indicate directions horizontally towards or away from the screen basket.

The instruments may be surgical instruments and other medical equipment in particular.

The screen basket positioning element 1 can be arranged on the screen basket. In the example, the screen basket positioning element 1 can be arranged on an upper edge section of the screen basket.

It may be possible for the screen basket positioning element to be designed in one piece with the screen basket instead.

The screen basket positioning element 1 can be made of plastic or metal. It is possible that the screen basket positioning element 1 is made partly of plastic and/or partly of metal. Preferably, the screen basket positioning element 1 is an injection-molded part made of plastic.

The screen basket positioning element 1 has a first mechanical interface 2, which is designed to restrict movement of the screen basket in the sterile goods container. The first mechanical interface 2 is an interface to the sterile goods container. In the example, the first mechanical interface 2 comprises contact surfaces to reduce play between the screen basket and the sterile goods container. In the example, the contact surfaces are arranged on at least one outer edge area of the screen basket positioning element 1. In the example, three outward-facing contact surfaces are provided. It may be provided that the first mechanical interface 2 comprises one, two or more than three contact surfaces.

The screen basket positioning element 1 also comprises two upward-facing surfaces 3. These surfaces 3 can be provided for stacking the screen basket with another screen basket. The screen baskets can be of different heights. The upper screen basket can be placed on the surfaces provided on the lower screen basket for stacking.

In the example, the screen basket positioning element 1 has two corner pieces and a web that connects the two corner pieces.

In one embodiment, the screen basket positioning element 1 has at least one flushing channel 4, which leads through the screen basket positioning element 1. In the example, four spaced flushing channels are provided for each corner piece. In the example, the flushing channels are designed as slotted holes. The flushing channels can also have a different shape or position. More or fewer than four flushing channels can also be provided.

In the example, a first machine-readable interface 5 is printed on at least one of the surfaces 3. It is also possible that the first machine-readable interface 5 is embossed into this surface 3. In addition, the machine-readable interface 5 can be generated during an injection molding process of the screen basket positioning element 1.

In one embodiment, the first machine-readable interface 5 comprises an optically readable marker that machine-readably encodes at least one type of the screen basket. The optically readable marker is, for example, a barcode or a data matrix code or a QR code. In one embodiment, the optically readable marker comprises alphanumeric data.

In one embodiment, the first machine-readable interface 5 comprises a transmitter. The transmitter is designed to send an electromagnetic signal to detect the screen basket. The electromagnetic signal is, for example, a Bluetooth Low Energy signal or a Radio Frequency Identification signal.

The transmitter can be glued to the screen basket positioning element 1 or fixed in a force-fit or form-fit manner. It may be provided that the screen basket positioning element 1 is at least partially made of plastic and the transmitter is surrounded by plastic.

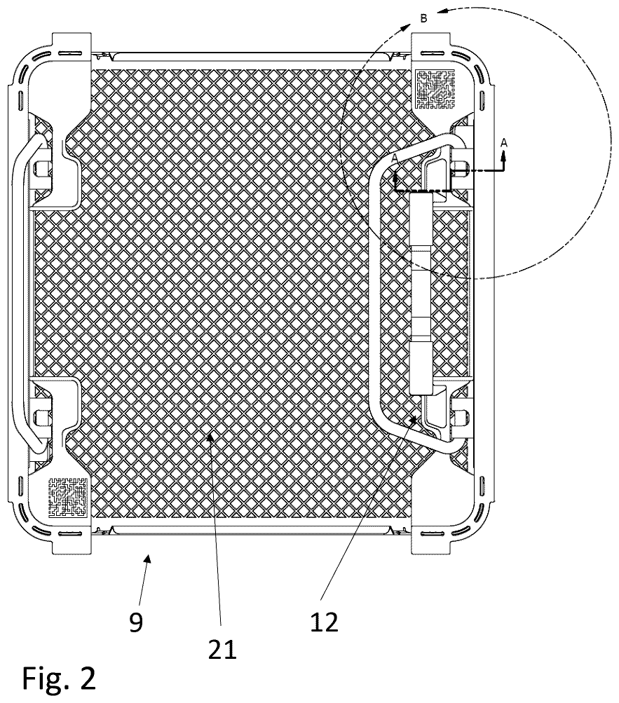

The screen basket positioning element 1 has a protective chamber 11, which is partially bounded by a partition wall 12. Protective space 11 is separated from a storage space 21 shown in FIG. 2 by partition 12. In storage room 21, instruments to be sterilized or sterile goods are flushed with sterilizing fluid and thus sterilized.

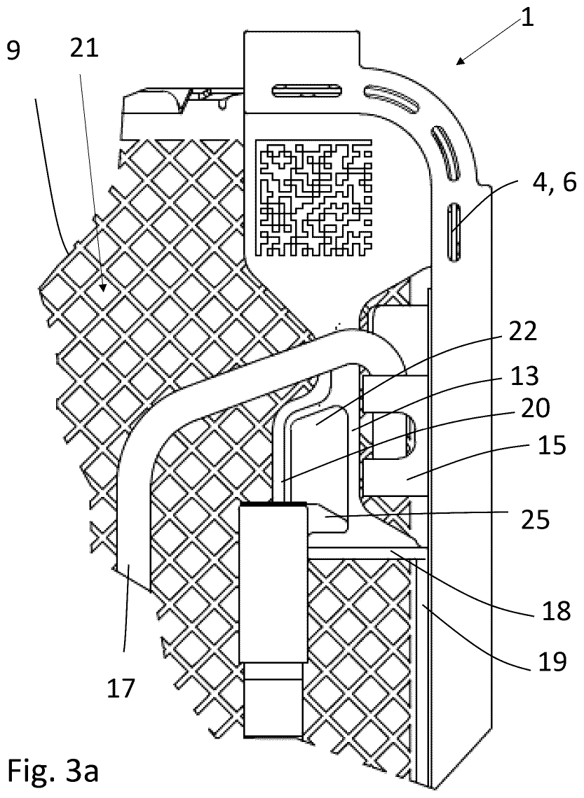

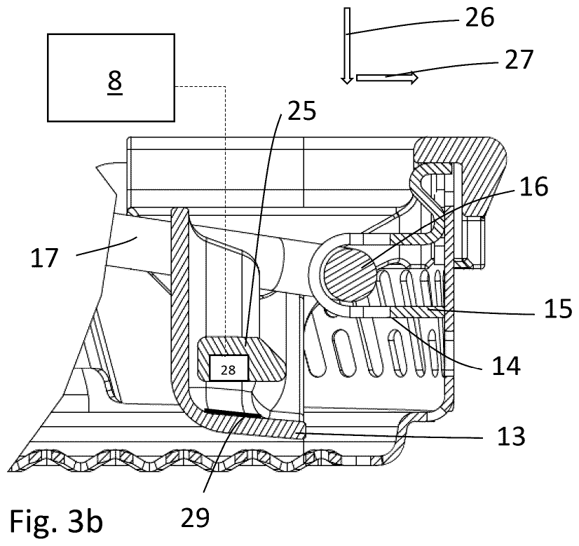

A multi-dimensionally complex shaping of the partition wall 12 is explained below by means of a synopsis of FIGS. 1 through 3b. FIG. 3b shows a detail of the screen basket 9 with one of the two screen basket positioning elements 1. FIG. 3b shows a detail from FIG. 2 along the stepped line A-A.

The partition wall 12 has lower wall parts 13, which are arranged below the lower edges 14 of bearing lugs 15 with respect to a horizontal flushing position of the screen basket 9. The respective lower wall parts 13 are spaced from the outer contours of the bearing lugs 15 in vertical projection onto the base surface away from the edge section 6 and in horizontal directions pointing inwards in such a way that the screen basket positioning element 1 can be slipped onto the edge section 6 without the lower wall parts 13 coming into contact with the bearing lugs.

These bearing lugs 15 are bent from a sheet of the screen basket 9 and pivotably receive two ends 16 of handlebars 17. The handlebars 17 are made of bent round steel.

The partition wall 12 also has intermediate partition wall parts 18, which are arranged between the bearing lugs 15. The intermediate partition wall parts 18 are connected to each other by means of a connecting wall 19.

The partition wall 12 also has inner partition wall parts 20 which, with respect to a horizontal flushing position of the screen basket 8, are spaced apart from the bearing lugs 15 in a horizontal direction pointing perpendicularly away from an edge section 6 of the screen basket 9 and towards the storage space 21 to such an extent that horizontal arms 22 of at least one gripper 25 can dip below the lower edges 43 of the bearing lugs 15.

The inner partition wall parts 20 have recesses 23 in which the handlebar 17 is partially received and supported on the inner partition wall part 20 when the handlebar 17 is in a horizontal position.

The partition wall 12 has a lateral connecting part 24 which connects one of the inner partition wall parts 20 to one of a plurality of surfaces 3 on which a further screen basket can be deposited.

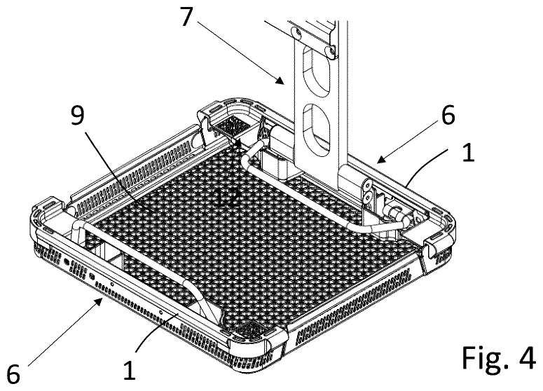

FIG. 4 shows a perspective view of an exemplary screen basket 9 with two screen basket positioning elements 1. In the example, two identical screen basket positioning elements 1 are provided. In the example, the screen basket positioning elements 1 are arranged on the opposite edge sections 6 of the screen basket 9.

In the example, one screen basket positioning element 1 is arranged on each side of the screen basket 9. The corner pieces are each arranged at a corner of the screen basket 9.

In the example, the screen basket 9 has a square base. In an alternative embodiment with unequal side lengths and a rectangular base, the screen basket positioning elements 1 are preferably attached to the shorter sides or the associated edge sections.

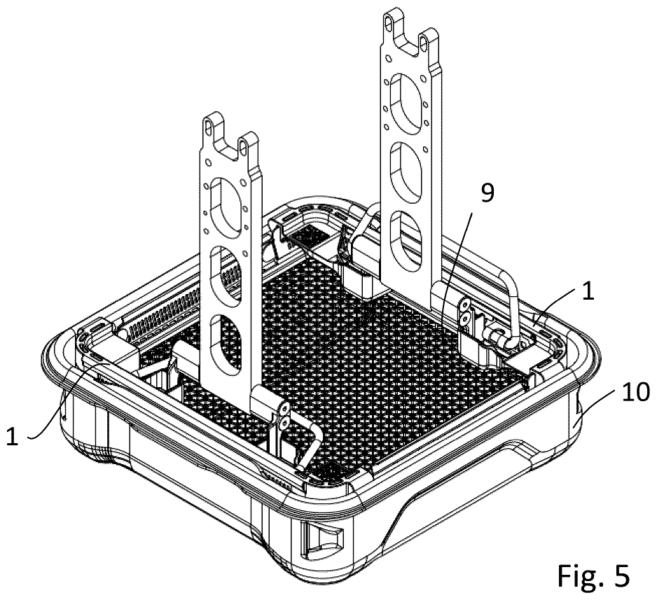

FIG. 5 shows a system comprising the screen basket 9 with two screen basket positioning elements 1 and the container 10. The container 10 comprises a trough for holding the screen basket 9. Screen basket 9 can be inserted into the tub of container 10 using the screen basket positioning element 1.

A method for gripping the screen basket 9 within container 10 is explained below. The arms 22 of the gripper 25 are moved vertically from above into the protective chamber 11, wherein the direction of movement is shown in FIG. 3b by means of an arrow 26. The arms 22 of the gripper 25 are then moved horizontally under at least one support part, which in the embodiment example is formed by the two bearing lugs 15. This direction of movement is shown in the drawing by means of a further arrow 27. Arms 22 of the gripper 25 grip behind or underneath the bearing lugs 15. The arms 22 are then raised again and come to rest against the undersides 14 of the bearing lugs 15, which thus form a support part for the arms 22.

In a continuous movement against the direction of movement of the arrow 26, the gripper 25 is then lifted with the arms 22 to lift the screen basket 9 out of the container 10.

The screen basket 9 is inserted into container 10 in the reverse order and with opposite directions of movement.

In an alternative embodiment of the method, the movement is not exactly vertical and horizontal according to the arrows 25, 27. Instead, the arms move at an angle or in an arc to insert the screen basket into the container and remove it again more quickly.

Moreover, the support part does not have to be the bearing lug 15. In this respect, only a single support part and/or a single arm can be provided.

An optical detection device 28 (FIG. 3b) can be coupled to the gripper 25, which detects an area within the protective space 11 in which a machine-readable marking 29 is located. An error is reported to the control device 8 if the machine-readable marking 29 cannot be detected or cannot be detected sufficiently. In this case, it is assumed that there is an object in the protected area. Detection device 28 is a camera and the machine-readable marking 29 is optically recognizable. The machine-readable marking 29 is located at the bottom of the protective space in a more horizontal position, so that this marking 29 is visible in a top view. This marking 29 enables control device 8, which is connected to the camera via a data line and an interface, to check whether objects are in the working or travel path of the gripper 25. This marking 29 is clearly formed as a defined pattern. If the pattern is not completely captured because there is an object in the working area, the automated gripping system does not move to a gripping position and reports an error to the control unit 8 via an interface. To be precise, the control device 8 causes the gripper 25:
not to move into the protective space 11; or
to stop a movement in the direction of the protective space 11; or
to move out of the protective space 11 again after the error is reported.

Each of the two lower wall parts 13 has its own machine-readable marking 29, which is printed or already applied to the surfaces of the lower wall parts 13 by an injection molding process. Alternatively, the marking 29 can also be applied by machining or by a laser ablation process.

Contrary to the embodiment example explained above, instead of a single protective space per screen basket positioning element 1, two protective spaces that are as small as possible and not connected to each other can also be provided in order to keep the storage space as large as possible. This provides more space for surgical instruments and other equipment.

1. A screen basket comprising:
a screen basket body; and
a screen basket positioning element,
the screen basket forming a storage space for flushing sterile goods,
the screen basket also forming a protective space within which a gripper is engageable to grip behind a supporting part,
the protective space being separated from the storage space by a partition wall, and
the screen basket positioning element being configured for positioning and facilitating gripping of the screen basket.

2. The screen basket according to claim 1, wherein the screen basket positioning element is plugged onto the screen basket body.

3. The screen basket according to claim 1, wherein the screen basket positioning element is attached to an edge portion of the screen basket body.

4. The screen basket according to claim 3, further comprising a support part and a handlebar with a first end and a second end, wherein:
the support part comprises a first bearing lug and a second bearing lug,
the first end of the handlebar is pivotably receivable in the first bearing lug, and
the second end of the handlebar is receivable in the second bearing lug.

5. The screen basket according to claim 4, wherein the partition wall has lower wall parts which, with respect to a horizontal flushing position of the screen basket, are each arranged below lower edges of the first and second bearing lugs and are spaced from outer contours of the first and second bearing lugs in horizontal directions pointing away from the edge portion and inwardly in such a way that the screen basket positioning element is placeable on the edge portion without the lower wall parts coming into contact with the first and second bearing lugs.

6. The screen basket according to claim 5, wherein the partition wall has intermediate partition wall parts arranged between the first and second bearing lugs.

7. The screen basket according to claim 6, wherein the intermediate partition wall parts are connected to one another by a connecting wall.

8. The screen basket according to claim 5, wherein the partition wall has inner partition wall parts which, with respect to a horizontal flushing position of the screen basket, are spaced from the first and second bearing lugs in a horizontal direction pointing perpendicularly away from the edge portion and towards the storage space to such an extent that the gripper is maneuverable below lower edges of the first and second bearing lugs.

9. The screen basket according to claim 8, wherein the inner partition wall parts have recesses in which the handlebar is partially received and supported on the inner partition wall parts when the handlebar is in a horizontal position.

10. The screen basket according to claim 8, wherein the partition wall has at least one lateral connecting part that connects one of the inner partition wall parts to one of a plurality of surfaces on which a further screen basket is stackable.

11. The screen basket according to claim 5, wherein one of the lower wall parts has a machine-readable marking.

12. The screen basket according to claim 11, wherein the machine-readable marking comprises a pattern.

13. The screen basket according to claim 1, further comprising a machine-readable interface outside the protective space, the machine-readable interface configured for detecting a position of the screen basket.

14. A method for processing the screen basket according to claim 1, the screen basket comprising a support part and being in a container, the method comprising the steps of:
moving an arm of a gripper into the protective space and under the support part; and
lifting the arm to lift the screen basket out of a container.

15. The method according to claim 14, wherein the step of moving the arm of the gripper into the protective space and under the support part comprises moving the arm vertically downwards and then horizontally under the support part.

16. The method according to claim 14, further comprising the step of scanning the protective space with an optical detection device coupled to the gripper to detect a machine-readable marking.

17. The method according to claim 16, further comprising the step of reporting an error to a control device when the optical detection device does not capture the machine-readable marking.

18. The method according to claim 17, wherein, when the control device receives the error, the control device:
causes the gripper not to move into the protective space,
stops movement of the gripper toward the protective space, or
moves the gripper out of the protective space.

19. A system comprising:
the screen basket according to claim 1; and
a container comprising a trough for receiving the screen basket,
the screen basket being insertable into the trough and adapted such that a mechanical interface for restricting movement of the screen basket in the container is placeable against an inner wall of the trough.
